(19) United States
(12) Patent Application Publication
(10) Pub. No.: US 2026/0071255 A1
(43) Pub. Date: Mar. 12, 2026

(54) A NON-INVASIVE DEVICE AND METHOD FOR DETECTING RNA ASSOCIATED DISEASE

(71) Applicants: SHUBHENDRA SINGH THAKUR, Nagpur (IN); DEOVRAT BEGDE, Nagpur (IN)

(72) Inventors: SHUBHENDRA SINGH THAKUR, Nagpur (IN); DEOVRAT BEGDE, Nagpur (IN)

(21) Appl. No.: 19/109,093

(22) PCT Filed: Sep. 7, 2023
(86) PCT No.: PCT/IB2023/058840

§ 371 Date: Mar. 5, 2025

(30) Foreign Application Priority Data
Sep. 7, 2022 (IN) 202221012042

(51) Int. Cl. C12Q 1/6809; C12N 9/22; C12N 15/113; C12Q 1/28; C12Q 1/34

(52) U.S. Cl. CPC C12Q 1/6809; C12N 9/226; C12N 15/113; C12Q 1/28; C12Q 1/34; C12Y 101/03004; C12N 2310/20; C12Q 2600/118

(57) ABSTRACT

A non-invasive device and method for detecting RNA associated disease based on the activity of the biological sample. The device and method encompass passing of the biological samples through one or more stacked layers in the first section which in turn induced enzymatic activity that is visually monitored. Thus, the capacity of the sample to digest and disintegrate the layers is measured to determine the risk assessment. Further, the same biological sample trickles through the permeable enclosure to the unitized chambers for the analysis. This unitized chamber is further connected to a light tight compartment to quantify miRNA levels present in the biological sample for determining the type of RNA based disease. Thus, the single device herein provides dual functionality of risk assessment and disease detection with minimized amount of sample.

### CROSS-REFERENCE TO RELATED APPLICATIONS AND PRIORITY

The present application claims priority from the Indian provisional patent application, having application No. 202221012042, filed on 7 Sep. 2022 and PCT application no. PCT/IB2023/058840 filed on 7 Sep. 2023, incorporated herein by a reference.

### TECHNICAL FIELD

The present subject matter described herewith, in general relates to identification and assessment of proteolytic entities and RNA species in a biological fluid. More particularly, the present subject matter relates to a device and method for identification and assessment of enzymatic activity and RNA species in a biological fluid for screening and identification of disease associated onset RNA and severity.

### BACKGROUND

A genetic disease is any disease caused by an abnormality in the genetic makeup of an individual. A genetic disorder is a disease caused in whole or in part by a change in the DNA sequence away from the normal sequence. A person skilled in the art recognizes that genetic disorders can be caused by a mutation in one gene, by mutations in multiple genes, by a combination of gene mutations and environmental factors, or by damage to chromosomes.

Genetic disorders may also be complex, multifactorial, or polygenic, meaning they are likely associated with the effects of multiple genes in combination with lifestyles and environmental factors. Multifactorial disorders include cancer, heart disease and diabetes (hereinafter maybe alternatively referred to as “diabetes mellitus”).

Some of the diseases are caused by mutations in the genes that are inherited from the parents and are present in an individual from birth such as sickle cell anemia. Other diseases are caused by acquired mutations in a gene or group of genes that occur during a person's life. Such mutations are not inherited from a parent but occur either randomly or due to some environmental exposure. Examples of diseases that are caused by acquired mutations in a gene include but are not limited to cancer caused due to smoking and neurofibromatosis.

In all over the world, the incidence of genetically acquired disorders is the highest. There are more than 6,000 well known genetic disorders and around 65% of people have some kind of health problem as a result of genetic mutations. Most commonly known examples of genetic diseases include but are not limited to Huntington's disease, Sickle cell anemia, Cystic fibrosis, Phenylketonuria, Glycogen storage diseases, Galactosemia, Hemophilia and Hereditary spherocytosis.

In developed countries, it has come to the realization that genetic diseases are a major cause of disability, and also result in fatal outcomes such as prolonged hospitalization and even death of an individual. Due to the wide range of genetic disorders that are known, diagnosis is widely varied depending on the type of genetic disorder. Most genetic disorders are diagnosed pre-birth, at birth, or during early childhood and in the adulthood. By early assessment and detection of the genetic disorders, the genetic predominance can be avoided.

In everyday life humans are constantly exposed to many factors such as environmental factors and human-made factors that can cause genetic damage or result in change in genetic makeup of an individual and often influences certain gene expression without any traceable change in the DNA, this extra genetic regulation is called epigenetic control. The environmental factors bringing in genetic and epigenetic change may include viruses, compounds produced by plants, fungi, and bacteria, industrial chemicals, products of combustion, alcohol, ultraviolet and ionizing radiation.

The management of genetic disease can be divided into counseling, detection, diagnosis, and treatment. In brief, the fundamental purpose of genetic counseling is to help the individual or family understand their risks and options and to empower them to make informed decisions. Diagnosis of genetic disease is sometimes clinical, based on the presence of a given set of symptoms, and sometimes molecular, based on the presence of a recognized gene mutation, whether clinical symptoms are present or not. Although effective treatments exist for some genetic diseases, for others there are none.

There are instances where either there are no notable genetic mutations still there are disease related pathologies or despite having disease related genetic markers there are not any noticeable abnormalities throughout the life of an individual. Thus, it becomes apparent that the prediction of disease manifestation just on the basis of traceable genetic mutations is not a precise solution in the pre-symptomatic testing domain.

Besides, detection of RNA associated disorders such as cancer at early stages can significantly alleviate patient discomfort, improve prognosis, therapeutic intervention, survival rates, and recurrence. However, detection and monitoring of the disease often requires painful invasive procedures such as biopsies and repeated blood draws.

Thus, a number of widely used techniques for the detection of a particular RNA sample such as saliva-based microbial, immunologic, and molecular biomarkers, northern blot technique, nuclease protection assays (NPA), in situ hybridization, and reverse transcription-polymerase chain reaction (RT-PCR) are adapted as an alternative to bypass invasive measures to evaluate the stage, type, and intensity of disease.

Thus, the expression of any genetic mutation engraved in the DNA can be traced using the RNA profile and the RNA activity, a precise tool for early prediction of many disease conditions much before clinical symptomatic manifestation of the same.

In the state of the art, a US application ‘U.S. Pat. No. 9,353,409B2’ discloses methods and compositions having trehalose and DNA polymerase for facilitating the rapid and efficient amplification of nucleic acid molecules and the detection and quantitation of RNA molecules, and for increasing the detection sensitivity and reliability through generation of secure cDNA molecules prior to gene-specific primer dependent amplification. The reagent mixture comprises a ready to use reagent solution, wherein the solution comprises: (a) trehalose in a concentration between about 5% and about 35%; (b) a viral reverse transcriptase; and (c) at least one DNA polymerases, in a buffer suitable for use in a reverse transcription reaction, wherein the buffer comprises a co-factor metal ion and nucleoside triphosphates.

In the state of the art, another US application ‘U.S. Pat. No. 5,945,515A’ disclosed solutions and methods for the effective, simple isolation/extraction of DNA, RNA and proteins from a single biological material sample, such as cells, tissues and biological fluids. The preferred solutions include effective amounts of a chaotropic agent(s), buffer, reducing agent, and may or may not include an organic solvent. Genomic DNA and total RNA can be isolated utilizing the solutions and methods of the invention in as little as 20 minutes, and proteins in as little as 30 minutes.

In the art, the US application ‘US20090215102A1’ disclosed a method to assess breast cancer. Although the method only determines the risk of having a disorder, it fails to disclose the method of identification of a particular disorder.

Such technologies for the detection of RNA associated genetic disorders such as cancer includes RNA extraction from samples, amplification of the genome via an enzymatic process, followed by cDNA preparation and real-time PCR. However, such technologies are time consuming and require an expensive real-time detection enabled thermal cycler and extensive preprocessing of biological samples to enrich the target RNA preparation. Furthermore, these technologies require a substantial volume of samples and the use of large instrument setup and multiple equipment, consequently elevating the risk of errors.

Moreover, State-of-the-Art Laboratory equipment is thus a prerequisite for such RT-PCR based detection. Such conventional methods known in state of the art are expensive, time taking and are not available abundantly due to its economic burden.

Therefore, there is a long felt need to develop a critical device that provides dual functionality of determining the disease risk assessment and identification of disease associated onset RNA from a biological fluid in a sample, rapid and convenient way for screening and prediction of RNA associated disease onset. The present disclosure also solves the problem of providing a tool for the detection of target RNA with extreme specificity without requirement of its purification and amplification in a shorter duration.

### OBJECTS OF INVENTION

The principal object of the present disclosure is to provide a detection device and a method for determining risk assessment and detection of a type of an RNA associated disorder by analyzing a biological fluid.

Another object of the present invention is to provide a highly reliable detection tool for prognosis of a health condition of the user.

Yet another object of the present disclosure is to develop a device for detection of health condition and disease type by implementing an RNA based biomarker profiling.

Still, another objective of the present invention is to develop a device that employs an amplification free and extraction free method for detecting the RNA associated disorder.

Yet, another objective of the present invention is to develop a device that is cost effective, requires a minimum sample and expedites the diagnostic process in comparison to conventional methods.

### SUMMARY

This summary is provided to introduce concepts related to a device and method for detection of RNA species in a biological fluid. This summary is not intended to identify essential features of the claimed subject matter nor it is intended for use in determining or limiting the scope of the claimed subject matter.

In one embodiment, a non-invasive device for detecting RNA associated disease is disclosed. The non-invasive device comprises of a first section with a funnel central piece for determining the proteolytic activity of a biological sample, wherein the first section is casted with at least two stacked layers. The said device further may comprise a second section for identifying the proteolytic entities, and a target RNA species in the biological sample. Further, the said second section may comprise at least one unified chamber comprising an enzymatic mixture for identification, and cleaving of the target RNA species from the biological sample.

Further, the said device may comprise a light tight compartment comprising a fluorescent excitation source to yield fluorescence in response to presence of a target RNA species. The said device further may comprise of at least one permeable enclosure configured for trickling down the biological sample from the first section to the second section. The said permeable enclosure is enabled for passing the biological sample from the first section to the second section, if the biological sample digests at least two stacked layers of the first section.

In another embodiment, a method for detecting RNA associated disease by enabling the device is disclosed. The method includes various steps. The method may include a step of injecting a biological sample into an opening of the central funnel-like piece of a first section. The method may include another step of passing the biological sample in the first section to interact with each of the stacked layers, wherein the enzymes from the biological sample digests and disintegrates each of the stacked layers. The method may include further step of trickling down the biological sample in the at least one unitized chamber of a second section through at least one permeable enclosure. The method may include a step of reacting an enzymatic mixture present in the unified chamber with the target RNA species in the biological sample. The method may include a step of illuminating the unified chamber with a fluorescent excitation source of the light tight compartment to yield a chemiluminescent signal in response to a control, and a fluorescent signal in response to the presence of target RNA species.

Other features and advantages of the present invention will be apparent from the following detailed description of the invention which illustrates, by way of example, the principles of the invention.

### BRIEF DESCRIPTION OF THE DRA WINGS

The detailed description is described with reference to the accompanying figures. In the Figures, the left-most digit(s) of a reference number identifies the Figure in which the reference number first appears. The same numbers are used throughout the drawing to refer like features and components.

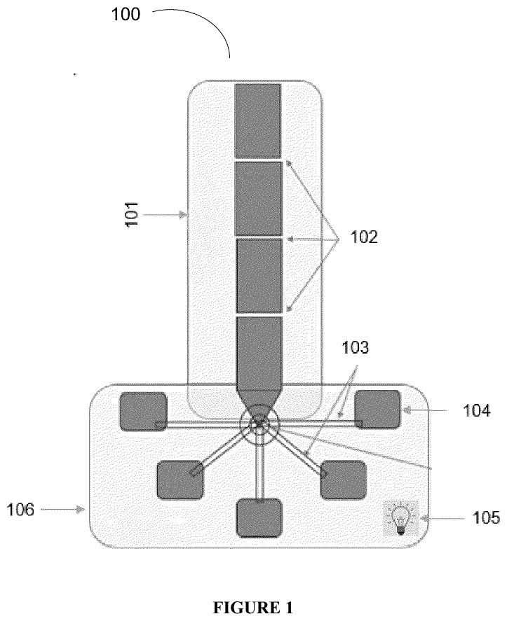

FIG. 1 depicts an isometric view of the non-invasive device (100) for detecting RNA associated disease, in accordance with an embodiment of the present disclosure.

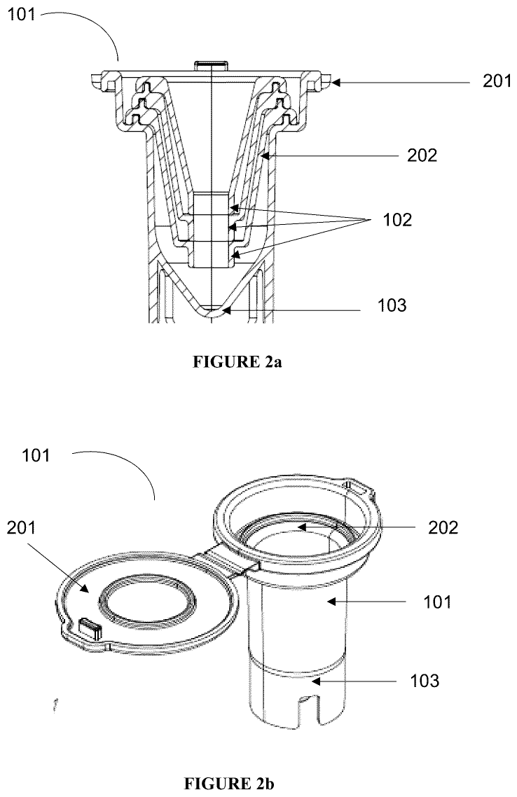

FIG. 2a depicts a cross sectional view of the first section (101) of the non-invasive device for detecting RNA associated disease, in accordance with an embodiment of the present disclosure.

FIG. 2b depicts an isometric view of the first section (101) of the non-invasive device (100) for detecting RNA associated disease, in accordance with an embodiment of the present disclosure.

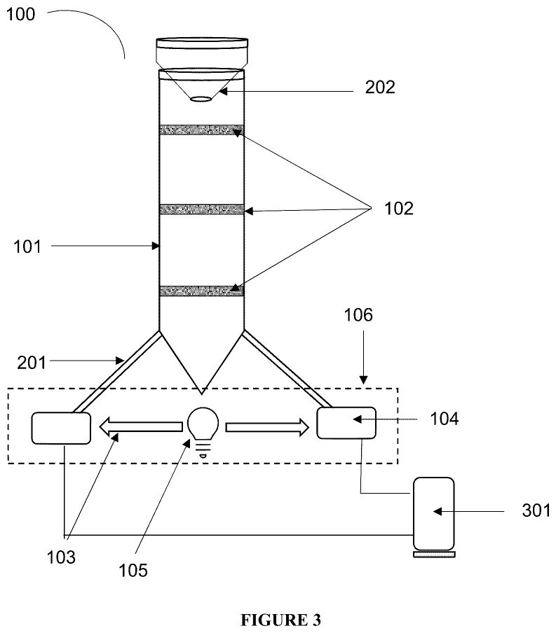

FIG. 3 depicts alternative arrangement of the non-invasive device (100) for detecting RNA associated disease, in accordance with an embodiment of the present disclosure.

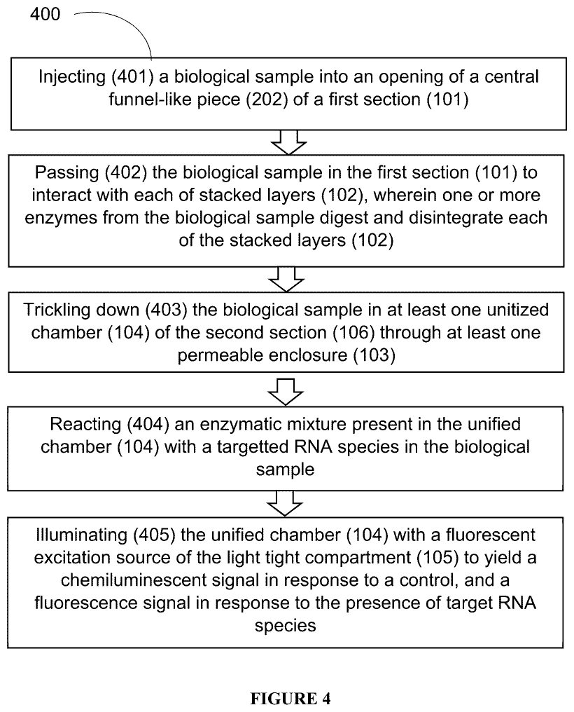

FIG. 4 depicts a flowchart of method (400) for detecting RNA based disease, in accordance with an embodiment of the present disclosure.

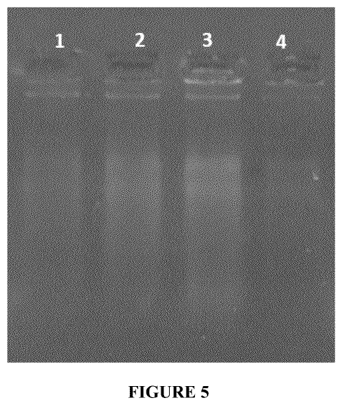

FIG. 5 depicts a pictorial representation of total RNA isolated from human saliva on 1% agarose gel, in accordance with an embodiment of the present disclosure.

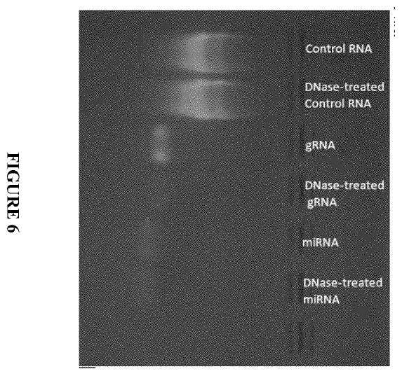

FIG. 6 depicts an In vitro transcription of miRNA and gRNA along with control RNA using template DNA from kit, in accordance with an embodiment of the present disclosure.

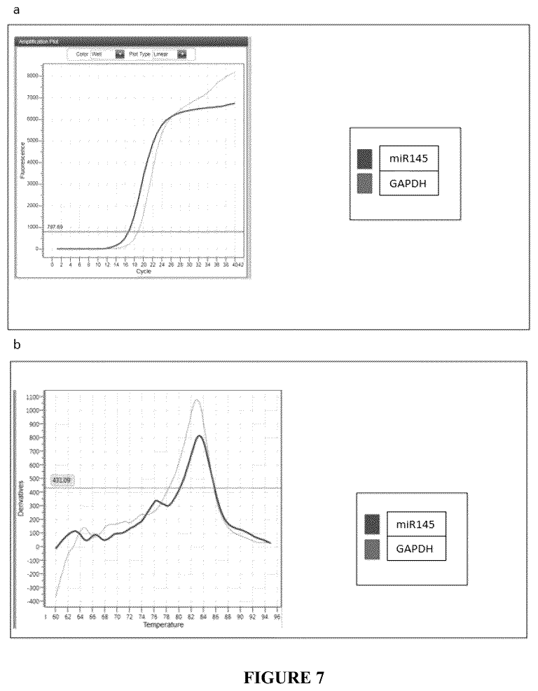

FIG. 7a depicts amplification plots of GAPDH (purple curve) and miR145 (green curve) RT-PCR using cDNA synthesized from RNA isolated from saliva, in accordance with an embodiment of the present disclosure.

FIG. 7b depicts melting curves of GAPDH (purple curve) and miR145 (green curve) RT-PCR using cDNA synthesized from RNA isolated from saliva, in accordance with an embodiment of the present disclosure.

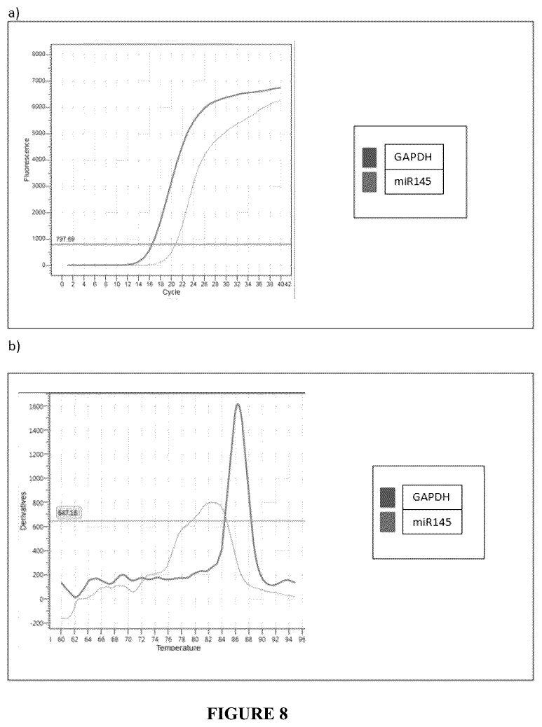

FIG. 8a depicts amplification plots of GAPDH (purple curve) and miR145 (green curve) RT-PCR using cDNA synthesized from direct saliva, in accordance with an embodiment of the present disclosure.

FIG. 8b depicts melting curves of GAPDH (purple curve) and miR145 (green curve) RT-PCR using cDNA synthesized from direct saliva, in accordance with an embodiment of the present disclosure.

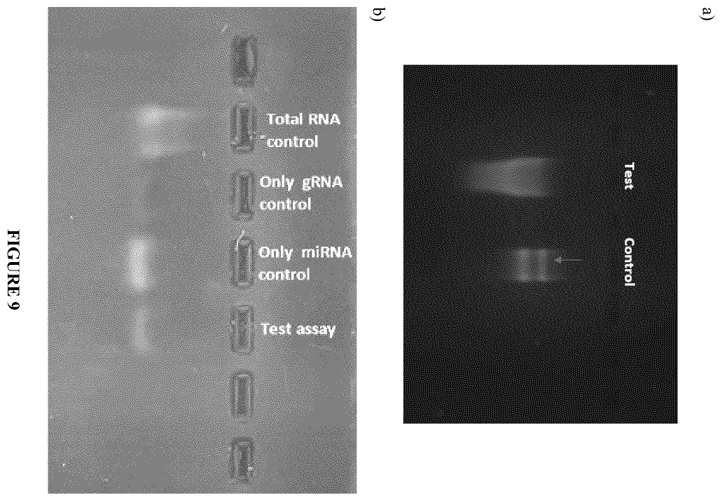

FIG. 9a depicts Cas13 assay pilot run with test and control assay, in accordance with an embodiment of the present disclosure.

FIG. 9b depicts a Cas13 assay pilot run with gRNA, miRNA and total RNA controls, in accordance with an embodiment of the present disclosure.

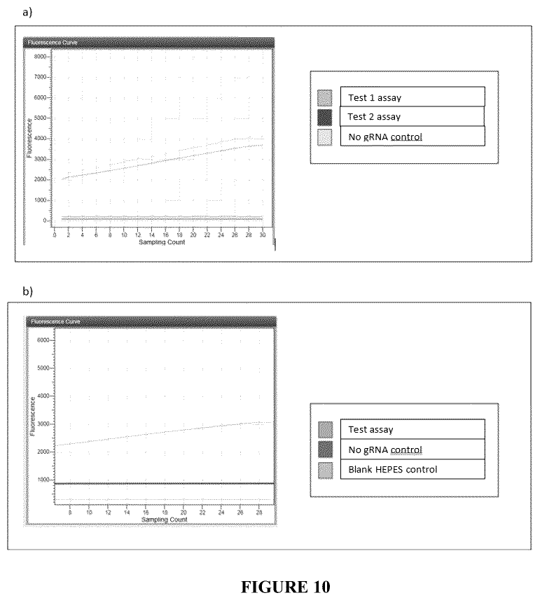

FIG. 10a depicts Cas13 assay performed with two different concentrations of Cas13 (Test 1: 1 μM; Test 2:0.8 μM) and standard miRNA target, in accordance with an embodiment of the present disclosure.

FIG. 10b depicts Cas13 assay was performed with 1 μM Cas13 and direct saliva target, in accordance with an embodiment of the present disclosure.

### DETAILED DESCRIPTION

Reference throughout the specification to “various embodiments,” “one implementation”, “some embodiments,” “one embodiment,” or “an embodiment” means that a particular feature, structure, or characteristic described in connection with the embodiment is included in at least one embodiment. Thus, appearances of the phrases “in various embodiments,” “in some embodiments,” “in one embodiment,” or “in an embodiment” in places throughout the specification are not necessarily all referring to the same embodiment. Furthermore, the features, structures or characteristics may be combined in any suitable manner in one or more embodiments.

Various modifications to the embodiment may be readily apparent to those skilled in the art and the generic principles herein may be applied to other embodiments. However, one of ordinary skill in the art may readily recognize that the present disclosure is not intended to be limited to the embodiments illustrated but is to be accorded the widest scope consistent with the principles and features described herein.

In state of the art, analysis of a biological sample (interchangeably referred to as biological fluid, body fluid, bodily fluids, biological cancer) is performed for the detection of RNA based diseases such as diabetes, cancer, Huntington's disease, Sickle cell anemia, Cystic fibrosis, Phenylketonuria, Glycogen storage diseases, Galactosemia, Hemophilia and Hereditary spherocytosis etc.

A non-invasive device (100) for detecting RNA associated disease is illustrated herewith, in accordance with an embodiment of the present disclosure.

In one embodiment, an enzymatic composition to identify any particular target RNA species and thereby a type of a disease in the said bodily fluid or biological fluid is disclosed herewith, in accordance with an embodiment of the present disclosure.

The said enzymatic composition may comprise a predetermined amount of enzyme such as glucose oxidase and/or Cas13a. The said enzymatic composition further may comprise a predetermined amount of peroxidase. The said enzymatic composition further may comprise predetermined amount of luminol (for detecting peroxidase activity in response to presence of glucose in the sample as control).

As used herein, the biological sample includes but is not limited to salivary secretions, semen, vaginal fluids, mucus, nasal secretions, sweat, pancreatic juice, gastric secretions and urine. However, saliva may be more preferred as saliva contains a variety of molecular and microbial analytes that are effective indicators of local and systemic disorders. Moreover, the proteomic and transcriptomic biomarkers relevant to cancer have been recently identified in the saliva.

As used herein, the term “body fluids” described herein may be any standard test sample comprising matrix metalloproteinases, biopsy sample, biological fluid, body fluid, saliva sample, urine sample, uterine sample, body tissue, swab, a blood sample or any pathological fluid sample, a physiological fluid sample, including, interstitial fluid, sweat, milk, ascites fluid, mucous, tissue extracts, cellular samples and the like.

As used herein, the proteolytic enzyme also known as protease, peptidase or proteinase such as collagenases, gelatinases and proteinases have unique property that catalyzes proteolysis, i.e., such enzymes are characterized for digesting, degrading and breaking down of proteins into smaller polypeptides or single amino acids. A specific group of 24 proteases, collectively called as matrix metalloproteinases (MMPs) are composed of zinc dependent endopeptidase that degrades proteins.

As used herein, CRISPR array is a family of DNA sequences found in the genomes of prokaryotic organisms such as bacteria and archaea. These sequences are derived from DNA fragments of bacteriophages that had previously infected the prokaryote. They are used to detect and destroy DNA from similar bacteriophages during subsequent infections as an adaptive immune response against the viruses.

As used herein, Cas13a is an RNA-dependent Rnase that can be programmed to bind an approximately 20 nucleotide long target RNA sequence with high specificity by designing a crRNA (CRISPR-RNA) or gRNA (guide-RNA) complementary to its target RNA sequence.

Cas13 enzymes target single-stranded RNA (ssRNA), with their signature effectors being Cas13a, Cas13b, Cas13d, Cas13X, and Cas13Y. Type VI CRISPR-Cas13 systems have an RNA-guided Rnase domain which when activated induces promiscuous collateral degradation of nearby ssRNA molecules. Upon binding and cleaving of a target, Cas13a develops an unspecific collateral Rnase activity. Cas13 enzymes have been employed for various CRISPR-based next generation diagnostic applications by exploiting highly specific target recognition and cleavage by Cas enzymes, followed by cleavage of reporters in trans by collateral activity.

In one embodiment, RNA may include but not limited to transfer RNA (tRNA), ribosomal RNA (rRNA), microRNA (miRNA), small interfering RNA (siRNA), small nucleolar RNA (snoRNA), Piwi-interacting RNA (piRNA), tRNA-derived small RNA (tsRNA), and small rDNA-derived RNA (srRNA), long non-coding RNA (lncRNA) and messenger RNA (mRNA).

As used herein, the term ‘miRNA’ described herein may be a key molecular component in the tumorigenic process, affecting several cell-signaling pathways essential to carcinogenesis. miRNAs are detectable in saliva and have shown potential as non-invasive biomarkers for a number of cancers including breast, oral, and lung cancers.

As used herein, a term “Unified chamber” refers to a single, integrated space or compartment that combines different functions or elements into a cohesive whole.

More particularly, the subject matter of the present invention illustrates a non-invasive device (100) enabled for detecting RNA associated disease risk which includes assessment and identification of a type of disorder by analyzing a biological sample. In one embodiment, the device (100) may comprise two main sections (101,106) such as a first section (101) for disease risk assessment and a second section (106) identification of RNA based disorder type detection. The second section (106) encompasses a CRISPR/Cas13-based enzymatic composition for detection of specific miRNA biomarkers in oral cancer. In one embodiment, specific miRNA biomarkers for oral cancer, more preferably miR145 present in the salivary samples was used. Furthermore, the proposed device (100) is compared with the gold standard method of RT-PCR and comparisons between isolated RNA and direct saliva has been shown.

In one embodiment, referring to FIGS. 1 & 2, the device (100) is configured for risk assessment, and detection/identification of RNA associated with a disease by analyzing the biological sample. Further, the device (100) comprises a first section (101) to determine a level of risk of a disease or a level of severity of the disease. Further, the first section (101) has a funnel central piece (202) that may be casted over or with one or more stacked layer (102), preferably at least two layers (102), more preferably three layers (102) stacked one over another.

In one embodiment, the central funnel-like piece (202) of the first section (101) may comprise of an inlet end configured to receive a biological sample and an exit end configured to drain out the biological sample towards a first layer (102).

In related embodiment, the each of the stacked layers (102) (interchangeably may be referred to as gelatin layer) casted herein are composed of at least one of albumin, gelatin, fibrin, and globin, more preferably gelatin. The analysis in first section (101) (interchangeably may be referred to as disease risk assessment assembly) is based on gelatinase activity of proteases enzyme present in the biological samples, preferably saliva samples comprising MMPs to digest and disintegrate the gelatin layers present in the first section (101). The gelatinase activity of the saliva of the subjects may also provide the detection and quantification of the RNA species onset of disorder state of an individual. The disorder state of an individual is any of diabetic state, cancerous state, or any other genetically impacted disorder of an individual glucose peroxidase.

Based on the efficiency of proteolysis and the composition domains in the family are divided into four main classes: Gelatinase (MMP-2, MMP-9), collagenase (MMP-1, MMP-8, MMP-13), stromelysins (MMP-3, MMP-10, MMP-12) and membrane metalloproteases (MT1-MMP, MT2-MMP, MT3-MMP, MT4-MMP). A typical MMP has a multi-domain structure including signal peptide, a prodomain, a catalytic domain, a hinge region and hemopexin like domain. The MMP-2 and MMP-9 are secreted gelatinases, also known as type IV collagenases. These enzymes mainly degrade collagen type IV, an important scaffold for the basement membrane proteins.

In state of the art, matrix metalloproteinases (MMP) and disintegrants are a family of proteolytic enzymes primarily raised in saliva secretions of subjects. The present disclosure provides a prognosis tool for assessment of proteolytic activity of enzyme matrix metalloproteinases (MMP) such as MMP-9 and MMP-2.

As used herein, the matrix metalloproteinases (MMP) may comprise but not limited to the collagenases such as MMP-1, MMP-8, and MMP-13, gelatinases such as MMP-2, and MMP-9, stromelysins such as MMP-3, MMP-10, and MMP-11, enamelysin such as MMP-20, matrilysin such as MMP-7, and MMP-26, metalloelastase such as MMP-12, membrane type MMPs, and other MMPs such as MMP-19, MMP-21, MMP-23A, MMP-23B, MMP-27, and MMP-28.

In another embodiment, referring to FIG. 1, the device (100) may comprise the second section (106) for identifying the proteolytic entities and target RNA species (interchangeably referred to as targetted RNA, target RNA) in the biological sample. Herein, the biological sample is transferred to the second section (106), if only the enzymes in the sample showed adequate proteolytic activity to digest at least three stacked layers (102). The second section (106) comprises at least one unified chamber (104).

Herein the unified chambers (104) comprising an enzymatic mixture are configured to collect the trickled biological samples from the first section (101). The enzymatic mixture facilitates the information on the type of disorder based on sequencing in RNA. Also, unlike the other conventional method, the present disclosure of the device (100) may be performed directly on a biological sample without any requirement of RNA isolation from the biological sample.

The enzymatic mixture is selected from the CRISPR/Cas13a reaction mixture, glucose oxidase-peroxidase and combination thereof. Herein, the CRISPR/Cas13a reaction mixture consisted of chloride salt, sulfonic acid buffering agent, Reporter RNA (rRNA), guide RNA (gRNA), and Cas13a.

More preferably, the CRISPR/Cas13a reaction mixture in the second section (106) may comprise gRNA in the range of 20 nM-5 μM; Cas13 type enzyme in the range of 80-120 nM; chloride salt in the range of 3-6 mM; fluorescent reporter in the range of 1 μM-250 nM; and sulfonic acid buffering agent in the range of 0.01-0.2 M.

Herein, the sulfonic acid buffering agent is more preferably HEPES (N-2-hydroxyethylpiperazine-N-2-ethane sulfonic acid). Herein, the assay buffer optimizes and adjusts the composition to minimize the required quantity of Cas 13a, thereby achieving improved efficiency compared to conventional methods.

In one embodiment, the unprocessed biological sample in the unified chamber (104) contains the microRNA as biomarker for disease. On detecting the presence of miRNA, the gRNA in the unified chamber (104) activates and guides Cas13a to the target miRNA for binding thereby leading to cleavage of target miRNA. Also, the Cas13a upon its activation by presence of target miRNA subsequently starts to cleave reporter RNA via its collateral activity.

Further, the said device (100) may comprise at least one permeable enclosure (103) connecting the first section (101) and the second section (106). The permeable enclosure (103) is configured for transferring down the bodily fluid from the first section (101) to the unified chambers (104) of the second section (106). Herein, the permeable enclosure (103) enables the transfer of the bodily fluid from the first section (101) to the second section (106) solely, if the body fluid digests at least three stacked layers (102) of the first section (101).

In one embodiment, the permeable enclosure (103) is selected from at least one of but not limited to the group of capillaries, a tubular passage, a sieve, a filtration membrane, a tubular opening with a bottom screw, a perforated structure, and a porous sheet, more preferably capillaries, or a tubular opening with a bottom screw.

In one embodiment, the permeable enclosure (103) is positioned between the first and second section (101, 106) to enable simultaneous risk assessment and disorder type detection from a single biological sample in a same device (100). This arrangement allows one to easily remove and discard the first section (101) in case the gelatin layer (102) is not effectively digested by the enzymes present in the biological sample. Herein, the first section (101) is detachably attached to the second section (106).

In another embodiment, referring to FIG. 3, the device (100) may further comprise a plurality of permeable enclosure (103) in form of capillaries placed externally alongside the first section (101).

In one embodiment, the permeable enclosure (103) connected to the first section (101) is configured to trickle down a portion of a biological sample to the unitized chambers (104). The unitized chambers (104) of the second section (106) work on a principal of a CRISPR/Cas13a enzyme-based detection of specific type of RNAs and thereby a biomarker related to a specific type of a disease.

In one embodiment, the unitized chambers (104) may comprise CRISPR/Cas13a reaction mixture. The Cas13a is an RNA-dependent Rnase that can be programmed to bind an approximately 20 nucleotide long target RNA sequence with high specificity by designing a crRNA (CRISPR-RNA) or gRNA (guide-RNA) complementary to its target RNA species sequence. Upon binding and cleaving of a target, Cas13a develops an unspecific collateral Rnase activity.

In one embodiment, the unitized chambers (104) may comprise luminol as a control, peroxidases to validate and measure enzyme detection, or glucose levels in the biological sample by detecting the hydrogen peroxide produced due to the glucose oxidation reactions. Herein, the luminol emits light when it reacts with hydrogen peroxide in the presence of peroxidase enzyme.

In another embodiment, the said device (100) further may comprise a light tight compartment (105) comprising a fluorescent excitation source including but not limited to UV LED. The collateral Rnase activity due to Reporter RNA enables production of fluorescence in response to presence of a cleaved biomarker RNA. The fluorescence may be quantified and assessed to detect a type of biomarker present in the biological sample and thereby a type of the disorder. The device (100) can also be customized to yield a chemiluminescent Control Signal, in response to biological samples such as but not limited to salivary glucose levels in one of the unitized chambers (104). Herein, the concentrations of glucose are used as control to verify that the device (100) and method are working correctly.

In one embodiment, the unitized chambers (104) producing a chemiluminescence may further be enabled to send a control signal to a display unit (301).

In another embodiment, the said device (100) further may comprise one or more optical fibers running out of every unitized chamber (104) (interchangeably referred to as unitization chamber) to transmit the fluorescent signal to a light-dependent resistor (not shown in figure) or a simple photodiode detection unit (not shown in figure).

In another embodiment, the said device (100) may optionally comprise an external or internal power source which may be used to combine the said simple photodiode detection unit to a radio transmitter (not shown in figure) for establishing a wireless connection and convenient readout on a display unit (301) including but not limited to mobile phone, computer etc.

In another embodiment, the said device (100) may comprise a cap (201) to cover the opening of the first section (101) in order to prevent entry of any impurities while testing, which may result in error.

In one embodiment, the device (100) may be employed as an ‘disease risk assessment kit’ detecting the proteolytic activity such as gelatinolytic activity of gelatinase and also a “disorder type detection kit” assessing the RNA biomarker species via the CRISPR/Cas13a based composition mixed with the biological sample.

As used herein, the device (100) may be interchangeably referred to as “kit”, “a testing apparatus”, “a detection apparatus”, “an activity detection apparatus”, “an enzyme detection apparatus”, “MMP protein enzyme detection apparatus”, “a proteolytic activity detection kit′, “a Gelatin-based Activity assay kit”, “a RNA detection device”, “a RNA detection apparatus”, a RNA detection unit” etc.

In one embodiment, the device (100) may comprise a central portion for visual monitoring of enzymatic activity (interchangeably referred to as gelatinase activity) and a light tight compartment (105) with unitized chambers (104) in close proximity to a UV LED as a fluorescent excitation source and an optical fiber running out of every CRISPR reaction unit to transmit the fluorescent signal to a light-dependent resistor or a simple photodiode detection unit.

In one embodiment, a device (100) comprising a plurality of unitization chamber (104) further may comprise different type of gRNA in the reaction mixture which may be used to identify particular RNA species in the given biological sample unique to the proposed solution and is not obvious to anyone skilled in RNA detection techniques. The different gRNA is incorporated to simultaneously detect different mRNA at same time, thereby determining different disease in a single device with limited available sample.

In one embodiment, a monitoring unit for determining an activity of the biological sample in the first section and second section for risk assessment and RNA associated disease identification. The activity herein may preferably include but not limited to:
proteolytic activity of the enzymes in the sample;
identification of the RNA-related diseases based on the illuminated light by the unified chamber;
Indication of proper function of the device (100) based on the detection of peroxidase activity within the unified chamber.

In another embodiment, a method (400) for detection of the RNA based disease based on a CRISPR/Cas13a RNA in a biological sample is illustrated in accordance with an embodiment of the present disclosure.

In related embodiment, the said method (400) comprises various steps as provided in FIG. 4. Referring to FIG. 4, the method (400) may comprise a step of injecting (401) a biological sample into an opening of a central funnel-like piece (202) of a first section (101). Herein, the biological sample is injected in the first section (106) in the range of 50 μl to 500 μl and preferably 100-200 μl.

The said method (400) further may comprise a step of passing (402) the biological sample in the first section (101) to interact with each of stacked layers (102). Herein, the enzymes present in the biological sample digest and disintegrate each of the stacked layers (102) by enzyme activity to migrate down the central funnel-like piece (202) of the first section (101).

In related embodiment, the said method (400) further may comprise a step of visual monitoring of the enzyme activity in the disease risk assessment assembly. The said method (400) further may comprise a step of simultaneous trickling of said biological sample through one or more permeable enclosure (103) to the respective unitized chamber (104), comprising CRISPR/Cas13a based enzyme composition enabled for the specific RNA detection.

In related embodiment, the said method (400) further may comprise a step of trickling down (403) the biological sample in at least one unitized chamber (104) of a second section (106) through at least one permeable enclosure (103). Further, the method (400) may include a step of reacting (404) an enzymatic mixture present in the unified chamber (104) with a target RNA species (interchangeably referred to as targetted RNA, target RNA) in the biological sample. Herein, the unified chamber (104) requires only 5-15 μl of the biological sample is for the enzymatic reaction.

The said method (400) further may comprise a step of illuminating (405) the unified chamber (104) with a fluorescent excitation source of a light tight compartment (105) to yield a chemiluminescent signal in response to a control, and a fluorescence signal in response to the presence of target RNA species.

In related embodiment, wherein the said CRISPR/Cas13a based enzyme composition with target RNA develops an unspecific collateral Rnase activity upon binding and cleaving of a target.

The fluorescence produced by the reaction of RNA from biological sample and the said enzyme may be detected and quantified, to determine a type of the disease/disorder. In one embodiment, the reaction may be monitored with the self-quenching fluorescent RNA beacon.

In one embodiment, the method (400) may further comprise a step of displaying (205) the disease risk assessment level and a type of a disease based on a collateral Rnase activity correlated with the CRISPR/Cas13a monitored with the self-quenching fluorescent RNA beacon or Reporter RNA.

In one example, as per the disclosure of the present invention the device (100) which serves as a disease type detection kit provides a preliminary estimate of a glucose level in body fluids and thereby preliminary analysis of diabetic state of an individual.

Also, the method only requires basic sample preprocessing, which is cost effective, easy to perform and requires a very minimum amount of sample. Therefore, the disease risk assessment and detection kit (100) are cheaper, sensitive and a better substitute for conventional RNA detection techniques. Further, for better understanding of the present disclosure and the associated method, following examples are discussed.

### Example 1: Biological Sample Activity in the First Section

### A: Standardization of MMP

In order to identify digestion property of the MMP, three different media were prepared. The three different media included Bovine Serum Albumin (BSA) 2% control, MMP9 with 2% BSA, and MMP9 without BSA. Further, the different media sample were used in the first section of the device and the observations regarding time required for digestion of layer were noted.

TABLE 1
Digestion on BSA CONTROL 2%

| No. of sets | Crossed 1^{st} layer |
|---|---|
| 1 | 11.55 min. |
| 2 | 12.43 min. |

TABLE 2
Digestion on MMP 9 WITH 2% BSA

| No. of sets | Crossed 2^{nd} layer |
|---|---|
| 1 | 14.3 min. |
| 2 | not crossed |
| 3 | not crossed |

TABLE 3
Digestion on MMP 9 WITHOUT BSA

| No. of sets | Crossed 2^{nd} layer |
|---|---|
| 1 | 8.4 min. |

It was noted from table 1, 2 & 3 that digestion of only first layer in the first section was observed for BSA control, whereas digestion of two stacked layers was observed using MMP 9 with 2% BSA and MMP 9 without BSA, indicating the activity of MMP 9.

Further, the MMP 9 and MMP 2 of different quantities were selected for testing the time required for the digestion on the layer.

TABLE 4
Digestion on MMP 9 & MMP 2 over the time

| | | | |
|---|---|---|---|
| | | Crossed 2^{nd} | Crossed 3^{rd} |
| KIT ASSAY | No of Sets | layer | layer |

| | |
|---|---|
| MMP 9 | 18% Gelatine 3 days drying at RT |

| | | | | |
|---|---|---|---|---|
| 25 ng | 1 | 9.11 | min | Not crossed |
| | 2 | 20.01 | min | Not crossed |

| | | | |
|---|---|---|---|
| 40 ng | 1 | Not crossed | — |
| | 2 | Not crossed | — |

| | |
|---|---|
| MMP 2 | 18% Gelatine 3 days drying at RT |

| 25 ng | 1 | not crossed | | — |
|---|---|---|---|---|
| | 2 | 18.46 | min | 20.44 min |
| 40 ng | 1 | 18 | min | not crossed |

| | | |
|---|---|---|
| 2 | not crossed | — |

| | |
|---|---|
| MMP 2 | 18% Gelatine 3 days drying at RT |

| | | | | |
|---|---|---|---|---|
| 25 ng | 1 | 18.33 | min | not crossed |
| | 2 | 19.00 | min | not crossed |
| 40 ng | 1 | 9.43 | min | not crossed |
| | 2 | 21.08 | min | not crossed |

| | |
|---|---|
| MMP 9 | 18% Gelatine 3 days drying at RT |

| | | | | |
|---|---|---|---|---|
| 25 ng | 1 | 2.02 | min | Not crossed |
| | 2 | 2.36 | min | Not crossed |
| 40 ng | 1 | 2.00 | min | Not crossed |
| | 2 | 2.14 | min | Not crossed |

| | |
|---|---|
| COMBINATION | 18% Gelatine 18 hours drying at RT |
| OF MMP 2 & 9 | |

| | | | | |
|---|---|---|---|---|
| 25 ng × 25 ng | 1 | 23.49 | min | Not crossed |
| (MMP 2 × MMP 9) | 2 | 11.03 | min | Not crossed |
| 40 ng × 40 ng | 1 | 16.04 | min | Not crossed |
| (MMP 2 × MMP 9) | 2 | 9.13 | min | Not crossed |
| 25 ng × 40 ng | 1 | 9.27 | min | Not crossed |
| (MMP 2 × MMP 9) | 2 | 10.53 | min | Not crossed |
| 40 ng × 25 ng | 1 | 3.20 | min | Not crossed |
| (MMP 2 × MMP 9) | 2 | 6.22 | min | Not crossed |
| MMP9 (80 ng) | 1 | 03.36 | min | 15.00 min |
| APMA activated | | | | |
| | 2 | 03.46 | min | 16.36 min |
| MMp9 (120 ng) | 1 | 7.22 | min | 13.40 min |
| APMA Activated | | | | |

It was concluded from table 4 that MMP9 and MMP2 when present together in the sample digests three stacked layers, indicating proteolytic activity and presence of tumorigenic cancers.

### B: Gelatin Kit as Internal Casted Layer Structures in the First Section

The gelatin-agarose kit is prepared by initially mixing 18% Gelatin powder (Bovine type B) with MMP buffer (50 mM Tris-HCl pH 7.6, 300 mM NaCl, 5 mM CaCl₂), 1 mM ZnCl₂, 20 mg % SDS), followed by the addition of 0.25% agarose. The mixture is gently heated until it is homogeneous, without boiling. The resulting liquid gelatin is made into thin films on the kit molds of varied sizes and left to dry overnight in a controlled environment. The dried kits are then carefully inspected and packaged, ensuring their quality and usability. Further, the stability and shelf life of the device (100) over the time are detected.

Table 5 (a-e): Activity of the First Section (101) of the Device (100) with BSA as Control And Trypsin as Experimental Group Over the Month

TABLE 5(a)
DAY 1

| | WITH AGAR | | | |
|---|---|---|---|---|
| No of sets | Crossed 1^{st} layer | Crossed 2^{nd} layer | Crossed 3 layer | |
| BSA | | | | |
| 1 | 4.14 min | not crossed | not crossed | |
| 2 | 3.31 min | 6.4 min | not crossed | |
| 3 | 5.12 min | not crossed | not crossed | |
| TRYPSIN 2.5% | | | | |
| 1 | 1.16 | 3.27 | 16.11 | min |
| 2 | 1.59 | 3.39 | 15.26 | min |
| 3 | 2.06 | 8.49 | 16 | min |

TABLE 5(b)
DAY 8

| Set | Crossed 1^{st} layer | Crossed 2^{nd} layer | Crossed 3 layer |
|---|---|---|---|
| | WITH AGAR | | |
| BSA | 56 sec | 3 min | not crossed |
| TRYPSIN | 1.09 min | 2.34 min | 14 min |
| | WITHOUT AGAR | | |
| BSA | 11.36 min | not crossed | — |
| TRYPSIN | 1.32 min | 6.56 min | 20.12 min |

TABLE 5I
DAY 19

| Set | Crosse^{d} 1st layer | | Crosse^{d} 2nd layer | | Crossed 3 layer |
|---|---|---|---|---|---|
| | WITH AGAR | | | | |
| BSA | 4.41 | min | 14.59 | min | not crossed |
| BSA | 13.25 | min | 17.06 | min | not crossed |
| TRYPSIN | 1.45 | min | 21.35 | min | not crossed |
| TRYPSIN | 1.40 | min | 3.53 | min | not crossed |
| | WITHOUT AGAR | | | | |
| TRYPSIN | 2.12 | min | 17.38 | min | not crossed |

| | | | | |
|---|---|---|---|---|
| BSA | 1.30 | min | not crossed | — |

TABLE 5(d)
DAY 25

| | WITH AGAR | | |
|---|---|---|---|
| Set | Crosse^{d} 1st layer | Crosse^{d} 2nd layer | Crossed 3 layer |
| BSA | 3 min | not crossed | — |
| TRIPSIN | 1.07 | 3 | 20 |
| TRIPSIN | 4.25 | 7.36 | 19.37 |

TABLE 5(e)
DAY 26

| No of sets | Cro^{ss}ed 1st layer | Cro^{ss}ed 2nd layer | Crossed 3 layer |
|---|---|---|---|
| | BSA | | |
| 1 | 12.17 | not crossed | — |
| 2 | 6.23 | 11 | 20 |
| 3 | 5.4 | 12 | 22 |
| | TRYPSIN | | |
| 1 | 3.44 | 6.18 | 12.38 |
| 2 | 3.1 | 11.05 | 19 |
| 3 | 2.35 | 4.37 | 12.3 |
| 4 | 3.49 | 12.52 | 19.47 |

### Example 2: Biological Sample Activity in the Second Section (106)

A: Sample Collection and Processing Along with Total RNA Isolation

The procedure for collecting saliva samples followed established guidelines to ensure the precise and consistent collection of samples. Patients were required to refrain from eating, drinking, and smoking for a minimum of 30 minutes before the collection, as this fasting period aids in minimizing potential contaminants in the saliva.

Unstimulated early morning saliva was gathered from patients at various stages of Oral Squamous Cell Carcinoma (OSCC). Patients were instructed to rinse their mouths with water for 30 seconds. Subsequently, 10 ml of normal saline solution was introduced into the mouth and swirled with agitation for approximately 1-2 minutes following which the unstimulated saliva was carefully collected into a 5 ml sterile collection tube pre-filled with a buffering solution. Part of the sample was centrifuged at 10,000 rpm for 10 minutes and the supernatant was discarded. Remaining pellet was resuspended in appropriate buffer for RNA isolation. Saliva samples were isolated from four subjects to ensure suitable extraction.

Further, total RNA was isolated from 5 ml normal human saliva using the Qiagen miRNEasy Mini Kit (Ref: 1038703) as per manufacturer's instructions. The isolated RNA was run on 1% agarose gel for 2 hours at 80V for qualitative and quantitative analysis.

B: In Vitro Transcription of Standard miRNA, gRNA, and GAPDH mRNA

The standard miRNA and corresponding gRNA were synthesized by in vitro transcription using T7 High Yield RNA Transcription Kit (Cat. No.: THY-50 rxn). Briefly, transcription templates with T7 promotor region attached were ordered for RNA synthesis in lab. 0.5 μg of dsDNA template of miRNA, gRNA, and GAPDH mRNA were transcribed using high yield T7 polymerase, 3 mM NTPs in standard buffer provided with the kit in a 20 μl reaction. The reaction was conducted at 37° C. for 16 hours. Post reaction, 10 μl of the prepared standard miRNA, gRNA was treated with 1 μl DNase I for removal of residual DNA and incubated for 15 min at 37° C.

C: Reverse Transcription for cDNA Preparation

cDNA synthesis was synthesized using the Qiagen miRNEasy Mini Kit (Ref: 1038703) and miRCURY RT SYBR. cDNA was synthesized from direct saliva samples, standard miRNA and total RNA for detection by RT-PCR. Direct saliva sample was processed by heating 10 μl of sample at 98° C. for 10 mins of which 2 μl reverse transcribed in a 10 μl reaction volume. 2 μl each of transcribed miRNA and total RNA were used for cDNA preparation in a 10 μl reaction volume. All further steps were followed as per the protocol provided on the kit.

### Example 3: Real-Time PCR Assay Conditions

RT-PCR for detection of miRNA in cDNA from direct saliva and standard miRNA was performed using miRCURY LNA RT (Ref: 339340) kit using locked nucleic acid primers targeting miR145. GAPDH primers were employed for amplification of endogenous control. The assay tube detecting standard miRNA included GAPDH cDNA as template for amplification of endogenous control. In case of direct saliva, no other cDNA was added. The conditions for RTPCR were as follows: −95° C.—5 min, 40 cycles of −95° C.—30 sec/−58° C.—1 min/−72° C.-01:30 min, and a final extension of −72° C.—10 min. The amplification plots and melting curves for the two assays were analysed.

### Example 4: CRISPR/Cas13a Assay Conditions

The Cas13 assays were performed with 1 μM LwaCas13a, 10 μM gRNA, 2 μM target miRNA-145, and 5 mM MgCl₂ in nuclease assay buffer (100 mM HEPES buffer, pH 7.4). LwaCas13a was pre-incubated with gRNA at 37° C. for 15 mins followed by the addition of 2 μg total RNA for pilot study and, the addition of 1250 nM fluorescent reporter RNA (FAM-based) with fluorescent kinetic measurement taken at an interval of 5 mins for 3 hours with excitation and emission range set at 493 nm & 517 nm. The detection of miRNA-145 was confirmed by fluorescence detection of the RNA biomarker and thereby the type of cancer was identified as oral cancer. Further, these findings were further implemented in casting the Cas13 assays (enzymatic mixture) in the second section of the disclosed device for detecting RNA associated disease.

### Example 5: Characterization Tests

A: Isolation of Total RNA from Saliva Sample

Total RNA isolated from the four saliva samples were subjected to agarose gel electrophoresis. Referring to FIG. 5, two distinct RNA bands were observed in the samples with respect to the varying concentrations.

B: In Vitro Transcription of Standard miRNA, gRNA Using T7 Polymerase

Using the T7 polymerase kit, in vitro transcription of standard miRNA and gRNA was performed along with the control RNA included in the kit. Any contaminating DNA was further removed by DNase I treatment. The in vitro transcribed RNA pre- and post-DNase I treatment were subjected to agarose gel electrophoresis (FIG. 6). The decreased intensity in the bands post treatment may be either due to removal of contaminating DNase or RNase contamination in the DNase I provided in the kit.

C: RT-PCR Based Detection of Standard miRNA from In Vitro Transcribed Template RNA And Direct Saliva Sample

The amplification plots and melting curves for miR 145 and GAPDH amplicons in the assay using standard miRNA template are depicted in FIG. 7a, b & FIG. 8a, b. The Ct values obtained with standard miRNA for endogenous control and miRNA were 16.3 and 18.8 respectively. The assay wherein direct saliva sample is used for cDNA preparation and RTPCR, Ct values for endogenous control and miRNA were 18.7 and 21.3 respectively. The melting curves, although show single peaks for the amplicons, are not very clean.

A difference of three cycles in Ct values in case of miRNA standard was observed between using isolated total RNA as template vs direct saliva sample as template. Single peaks in melting curves depict single product formation during the RT-PCR process thereby confirming the specificity of the primers. 1. The Ct values by using direct saliva were two to three cycles lower than that observed with standard miRNA template while the curve showed single peaks.

D4: Detection of In Vitro Synthesized miRNA Target with Total RNA for Collateral Activity Using CRISPR/Cas13 Assay

The in vitro test for Cas13 assay was initially carried out using total RNA degradation as a test for collateral activity. The test assay was run along with a control assay in which specific gRNA was not added. The lack of degradation of total RNA in control assay indicates that Cas13 was not activated by gRNA and target miRNA cleavage, though no collateral activity was detected (FIG. 9 a, b).

D5: Detection of In Vitro Synthesized miRNA Target Using Standard miRNA and Direct Saliva Sample, and Fluorescence Reporter for Collateral Activity Using CRISPR/Cas13 Assay

The Cas13 assay was conducted with standard miRNA as target and direct saliva sample using fluorescent probe for quantitation. Fluorescence values were quantitated at real time up to 3 h and graph was plotted (FIG. 10 a, b). It was observed that the fluorescence values ranged from 2000-4000 units where standard miRNA target was used in the assay while, using direct saliva sample for the assay gave a range of 2000-3000 units in the 3 h of analysis. It was observed that the detection of miRNA in the saliva was comparable to that in vitro although with slightly lesser sensitivity. The fluorescence readings tested indirectly through collateral activity were found to be relatively lesser in test assays conducted directly with saliva as compared to in vitro assays.

Thus, it can be concluded that the CRISPR/Cas13 assay was able to detect about 2 μM miRNA in assay as well as in the saliva. The detection of miRNA between control and test was found to be detectable i.e., a difference of 2000 U. The average miRNA concentration found in saliva is reported to be about 22 μM. The range of fluorescence units in standard miRNA assay ranged from 2000-4000 U in a span of 3 h while in direct saliva, it ranged from 2000-3000 U in a span of 0 h-3 h.

### Example 5: Method (400) for Identifying RNA Associated with Oral Cancer by Enabling The Device (100)

A 150 μl saliva sample is injected into the opening of the central funnel-like piece of the first section. Further, the biological sample is passed in the first section to interact with three gelatin layers. The enzymes in the biological sample digests and disintegrates each of the at least three gelatin layers which in turn leads to trickling the biological sample from one or more different unitized chambers second section through a permeable enclosure. Further, gRNA, reporter RNA and Cas13a in the enzymatic mixture (refer to table 6) reacted with the targeted RNA (specific miRNA biomarkers for oral cancer, more preferably miR145) of the biological sample trickled in in the unified chambers of the second section from the first section.

TABLE 6
composition of the enzymatic mixture

| Component | Stock | | Volume | Working Concentration |
|---|---|---|---|---|
| gRNA | 50 | μM | 2 μl | 5 uM, 10 μM, 50 nM, 20 nM |
| Cas13 | 1 | μM | 1 μl | 100 nM, 200 nM, 1 μM |
| MgCl₂ | 50 | mM | 1 μl | 5 mM |
| Fluorescent | 12.5 | μM | 1 μl | 1.25 μM, 125 nM, 250 nM |
| Reporter | | | | |

| | | | |
|---|---|---|---|
| HEPES buffer | 1M | 3 μl | 0.1M |

The second section is configured to detect the target miRNA in the biological sample when present in the range as low as 1 μM-20 nM. Further, at least one of the unified chambers is illuminated by a fluorescent excitation source from the light tight compartment (105) which results in yielding of the chemiluminescent control signal by component such as luminol and the fluorescence signal in response to the presence of target RNA thereby identifying the target RNA associated with oral cancer. The chemiluminescent control signal and the fluorescence signal in response to the presence of target RNA is then sent to the display unit via a detector unit i.e. fluorescent detection system. The total time required to carry out the detection in device 1 is less than 24 hours and preferably between 2-18 hours.

In accordance with embodiments of the present disclosure, the device (100) as described above may have following advantages including but not limited to:
The device (100) is a portable and inexpensive system.
The said device (100) is a reliable and precise tool for simultaneous disease risk assessment and detection of type of disease variety based on presence of RNA species in body fluids without any preprocessing of the same.
Unlike conventional RT-PCR-based detection, which typically requires 24-48 hours to yield results, this device (100) assay can deliver results within a timeframe ranging from 30 minutes to 10 hours.
The device (100) evaluates RNA-based species using a minimal sample size and shortened analysis time.
The device (100) operates by allowing the passage of the biological sample through the first section that will enabling the activation of the second section (106). This design effectively prevents sample wastage and resources of the second section (106).
The said device (100) provides a portable and handy tool for detection of specific disease and to simultaneously assess the risk level in a single device.

The foregoing applications of the device/kit (100) developed and disclosed herewith may include but not limited to RNA based disease type such as oral cancer identification, diabetic condition detection, virus type detection, gene expression studies, epigenetic analysis, therapeutic interventions, etc.

A person of ordinary skill in the art would understand the certain modifications could come within the scope of this disclosure. For limiting the scope of the disclosure and the present subject matter, a subsequent complete specification be filed. The true scope and content of this disclosure is to be determined in the subsequent complete specification.

1. A non-invasive device for detecting RNA associated disease, comprising:
a first section with a funnel central piece for determining the proteolytic activity of a biological sample, wherein the first section is casted with at least two stacked layers;
a second section for identifying proteolytic entities, and a target RNA species in the biological sample, wherein the second section comprises of:
at least one unified chamber comprising an enzymatic mixture for identification, and cleaving of the target RNA species from the biological sample, and
a light tight compartment comprising a fluorescent excitation source to yield fluorescence in response to presence of a target RNA species; and
at least one permeable enclosure configured for trickling down the biological sample from the first section to the second section,
wherein the permeable enclosure is enabled for passing the biological sample from the first section to the second section, if the biological sample digests the at least two stacked layers of the first section.

2. The device as claimed in claim 1, wherein the stacked layers of the first section are protein casted layers of at least one of albumin, gelatin, fibrin and globin.

3. The device as claimed in claim 1, wherein the permeable enclosure is selected from the group of at least one of capillaries, a tubular passage, a sieve, a filtration membrane, a tubular opening with a bottom screw, a perforated structure, and a porous sheet, more preferably capillaries, or a tubular opening with a bottom screw.

4. The device as claimed in claim 1, wherein the enzymatic mixture composition is selected from CRISPR/Cas13a reaction mixture, glucose oxidase-peroxidase, and combination thereof.

5. The device as claimed in claim 4, wherein the CRISPR/Cas13a reaction mixture of second section comprising:
gRNA in the range of 20 nM-5 μM;
Cas13 enzyme in the range of 80-120 nM;
chloride salt in the range of 3-6 mM;
fluorescent reporter in the range of 1 μM-250 nM; and
0.01-0.2 M sulfonic acid buffering agent.

6. The device as claimed in claim 1, wherein the first section is detachably arranged with the second section.

7. The device as claimed in claim 1, comprises a monitoring unit for determining an activity of the biological sample in the first section and second section for risk assessment and RNA associated disease identification.

8. A method for detecting RNA associated disease by enabling the device comprising the following steps:
injecting a biological sample into an opening of a central funnel-like piece of a first section;
passing the biological sample in the first section to interact with each of stacked layers, wherein one or more enzymes from the biological sample digest and disintegrate each of the stacked layers;
trickling down the biological sample in at least one unitized chamber of a second section through at least one permeable enclosure;
reacting an enzymatic mixture present in the unified chamber with a target RNA species in the biological sample; and
illuminating the unified chamber with a fluorescent excitation source of a light tight compartment to yield a chemiluminescent signal in response to a control, and a fluorescence signal in response to the presence of target RNA species.

9. The method as claimed in claim 8, wherein the biological sample is injected in the first section in the range of 100-200 μl, and 5-15 μl of sample is required for reaction in the unified chamber.

10. The method as claimed in claim 8, wherein the biological sample is trickled down in at least one unitized chamber if at least two stacked layers of the first section is digested.
